(19) United States
(12) Patent Application Publication
(10) Pub. No.: US 2020/0095506 A1
(43) Pub. Date: Mar. 26, 2020

(54) A PROCESS FOR PREPARING A PARAFFIN PRODUCT

(71) Applicant: SHELL OIL COMPANY, Houston, TX (US)

(72) Inventor: Parag KOTAK, Bangalore North, Karnataka (IN)

(21) Appl. No.: 16/495,697

(22) PCT Filed: Mar. 21, 2017
(86) PCT No.: PCT/EP2017/056616

§ 371 Date: Sep. 19, 2019

(30) Foreign Application Priority Data
Mar. 22, 2016 (EP) 16161563.8

(51) Int. Cl. C10G 2/00; C12P 7/06

(52) U.S. Cl. CPC C10G 2/30; C10J 2300/1665; C10J 2300/1659; C12P 7/065

(57) ABSTRACT

The invention relates to a process for preparing a paraffin product from a carbonaceous feedstock. The method comprises the steps of obtaining a gaseous mixture of carbon monoxide and hydrogen from a carbonaceous feedstock, performing a Fischer-Tropsch reaction and subjecting at least part of an off gas obtained from the Fischer-Tropsch reaction to fermentation.

### FIELD OF THE INVENTION

The present invention relates to a process for preparing a paraffin product from a carbonaceous feedstock.

### BACKGROUND TO THE INVENTION

The Fischer-Tropsch process can be used for the conversion of hydrocarbonaceous feed stocks into normally liquid and/or solid hydrocarbons (i.e. measured at 0° C., 1 bar). The feed stock (e.g. natural gas, associated gas, coal-bed methane, residual oil fractions, biomass and/or coal) is converted in a first step into a mixture of hydrogen and carbon monoxide. This mixture is often referred to as synthesis gas or syngas. The synthesis gas is fed into a reactor where it is converted over a suitable catalyst at elevated temperature and pressure into paraffinic compounds ranging from methane to high molecular weight molecules comprising up to 200 carbon atoms, or, under particular circumstances, even more.

The hydrocarbon products manufactured in the Fischer-Tropsch process are processed into different fractions, for example a liquid hydrocarbon stream comprising mainly C5+ hydrocarbons, and a gaseous hydrocarbon stream which comprises methane, carbon dioxide, unconverted carbon monoxide, unconverted hydrogen, and lower hydrocarbons. The gaseous hydrocarbon stream may also comprise nitrogen, argon and traces of olefins and oxygenates

The gaseous hydrocarbon stream is often referred to as Fischer-Tropsch off-gas. Fischer-Tropsch off-gas can be recycled to the syngas manufacturing or to the Fischer-Tropsch reactor. Sometimes lower hydrocarbons are removed before the off-gas is recycled. Lower hydrocarbons may be removed by decreasing the temperature of the off-gas and then applying a vapour-liquid separation. However, when the off-gas is recycled to the syngas manufacturing or to the Fischer-Tropsch reactor, the components in the off-gas which do not take part in the Fischer-Tropsch reaction, such as carbon dioxide, nitrogen and methane, occupy reactor space. The components which do not take part in the Fischer-Tropsch reaction are also referred to as “inerts”.

The level of inerts in the Fischer-Tropsch reactor increases with increasing Fischer-Tropsch off-gas recycling. The pace of the build-up of inerts can be reduced by treating the off-gas before it is recycled. One possibility is to recycle part of the Fischer-Tropsch off-gas to one or more Fischer-Tropsch reactors while another part of the off-gas is used as fuel. A downside of this is that only a part of the hydrocarbonaceous feed stock is converted to the desired C5+ hydrocarbons. When the off-gas is passed through a pressure swing adsorption unit (PSA), it is normally possible to remove carbon dioxide and water from the off-gas.

It is often possible to recover a hydrogen stream from the off-gas by means of a PSA unit; the hydrogen stream can be recycled to the Fischer-Tropsch reactor. Nevertheless, some common commercial PSA units do not or do not completely remove nitrogen from the off-gas and result a hydrogen stream that contains nitrogen. Additionally, common commercial PSA units are often not designed to recover a carbon monoxide stream. Therefore, it is common to recycle only a relatively small part of the off-gas.

There remains a need for improving the processing of off-gas from Fischer-Tropsch reactions.

### SUMMARY OF THE INVENTION

It has now been found that off gas from a Fischer-Tropsch reaction can be used in fermentation processes to obtain alcohols.

In an aspect the invention resides in a process for preparing a paraffin product from a carbonaceous feedstock comprising the following steps:

(a) Treatment of the carbonaceous feedstock to obtain a gas mixture comprising hydrogen and carbon monoxide;

(b) Performing at least once, a Fischer-Tropsch reaction using the mixture as obtained in step (a) and recovering a paraffin product and an off-gas comprising hydrocarbons, carbon dioxide, carbon monoxide, water and hydrogen and optionally, nitrogen, argon and traces of olefins and oxygenates, from the Fischer-Tropsch reaction; and

(c) Subjecting at least a part of the off-gas from the Fischer-Tropsch reaction, optionally after removing hydrocarbons, to anaerobic fermentation to obtain a fermentation product comprising alcohol.

### BRIEF DESCRIPTION OF THE DRAWINGS

FIG. 1 shows a non-limiting example of the present invention.

### DETAILED DESCRIPTION OF THE INVENTION

In the present disclosure with fermentation is meant the conversion of at least CO into alcohol by microbes. These microbes are capable of growing on CO-containing gases. Said microbes may or may not require sugar. Such a process is disclosed in US2011/0138684 which discloses a method of processing synthesis gas obtained from biomass by subjecting it to fermentation. It does not disclose fermentation based on off gas from a chemical reaction.

Off gasses have a different composition than synthesis gas. Off gasses have a lower carbon monoxide and hydrogen content and comprise other additional ingredients of which some may have, by presence or amount, a detrimental effect on the fermentation process. The off gas used in the present invention comprises hydrocarbons, carbon dioxide, carbon monoxide, water and hydrogen and optionally, nitrogen, argon and traces of olefins and oxygenates. The inventors found that an off gas comprising these ingredients can be processed with fermentation.

Said fermentation may be conducted in a bioreactor. The term bioreactor is meant to include devices such as a bubble column, trickle bed reactor (tbr), gas lift fermenter, continuous stirred tank reactor (cstr), immobilized cell reactor (icr), membrane reactor including hollow fibre membrane bioreactor (hfmbr), or a static mixer. Processes for the production of ethanol and other alcohols from gaseous substrates are known. Fermentation processes include those described for example in WO2007/117157, WO2008/115080, U.S. Pat. Nos. 6,340,581, 6,136,577, 5,593,886, 5,807,722 and 5,821,111.

In the present disclosure, with “a” and “an” is meant “at least one” unless indicated otherwise.

In step (a) of the process the carbonaceous feedstock is treated to obtain a gas mixture comprising hydrogen and carbon monoxide.

The treatment of the carbonaceous feedstock in step (a) is such that a gas mixture comprising carbon monoxide and hydrogen is obtained. This gas mixture is often referred to as syngas. Several methods are known for generating syngas from a carbonaceous feedstock. In an aspect of the invention the production of syngas from hydrocarbonaceous feedstock is effected by any one of gasification, partial oxidation, auto-thermal reforming, steam reforming, or any combination thereof.

In an aspect of the invention the gas mixture is obtained in step (a) by steam reforming and/or from the partial oxidation of natural gas. In an aspect of the invention partial oxidation is used form the production of synthesis gas from natural gas.

In an aspect the gas mixture is obtained by partial combustion of coal.

In a Fischer-Tropsch reaction a carbon monoxide and hydrogen containing gas mixture is contacted with a Fischer-catalyst in a reactor. Hence, in an aspect of the invention a step of contacting the gaseous mixture with a Fischer-Tropsch catalyst is included.

The Fischer-Tropsch reaction may be conducted in a fixed bed reactor, especially multi-tubular fixed bed reactor, fluidized bed reactor, such as entrained fluidized bed reactors and fixed fluidized bed reactor, and slurry bed reactor such as three-phase slurry bubble columns and ebulated bed reactors.

In case a fixed bed reactor is used, the Fischer-Tropsch reaction is preferably carried out at a temperature in the range from 125 to 400° C., more preferably 175 to 300° C., most preferably 200 to 260° C. The pressure preferably ranges from 5 to 150 bar, more preferably from 20 to 80 bar. The gaseous hourly space velocity may vary within wide ranges and is typically in the range from 500 to 10000 Nl/l/h, preferably in the range from 1500 to 4000 Nl/l/h. The hydrogen to CO ratio of the feed as it is fed to the catalyst bed generally is in the range of 0.5:1 to 2:1. In such a case a cobalt based catalyst is preferred.

Said off gas is used as a gaseous feedstock for fermentation. In an aspect of the invention at least part of the residual hydrocarbons present in the off gas are removed prior to subjecting the off gas to fermentation. Part of the residual hydrocarbons may be largely removed by subjecting the off gas to scrubbing in a scrubber or by cooling the off gas, for example to a temperature of 40° C. or less. In the scrubber the off gas is, optionally after cooling, contacted with a wash fluid comprising C5-C20 hydrocarbons. See for an example WO2014/013087. US2011306682 discloses another method of removing hydrocarbons from Fischer-Tropsch off gas. The method of US2011306682 includes absorbing light hydrocarbon compounds and a carbon dioxide gas from the off gas using an absorption solvent including liquid hydrocarbon compounds and a carbon dioxide gas absorbent, separating the absorption solvent which has absorbed the light hydrocarbon compounds and the carbon dioxide gas into the liquid hydrocarbon compounds and the carbon dioxide gas absorbent.

In an aspect the off gas obtained from the Fischer-Tropsch reaction comprises methane, carbon dioxide, carbon monoxide and hydrogen and optionally water, nitrogen, argon and traces of olefins and oxygenates. Preferably, the carbon monoxide content of the off gas is at least 5 vol % based on the total volume of the off gas. Preferably, the CO content is at the most 80 vol %. In an aspect of the invention the hydrogen content of the off gas is at least 5 vol %.

In an aspect of the invention the Fischer-Tropsch reaction is conducted at elevated pressure and the process comprises a further step prior to step (c) wherein the off gas pressure is lowered to a pressure of less than 10 bar, prior to subjecting said off gas to fermentation. This provides for a good fermentation in which at least 50% of the carbon monoxide is converted into alcohol. In an aspect of the invention the pressure is maintained within a range of 1 to 10 bar. In an aspect of the invention the off gas is subjected to a pretreatment step prior to step (c) in which the carbon monoxide level is adjusted such that the CO level is at least 20 vol %. A gas having a CO level of at least 20 vol % is suitable for use as a gaseous feedstock for fermentation.

The carbon monoxide and/or hydrogen level in the Fischer-Tropsch off gas may vary over time and may drop to levels below the level required to maintain the fermentation activity in the bioreactor. A drop in carbon monoxide level may for example be caused by a change in the composition of the carbonaceous feedstock or operational aspects such as a runaway of a Fischer-Tropsch reactor. In such cases carbon monoxide may be added to the off gas stream. In an aspect of the invention step (c) is performed in case the carbon monoxide level in the Fischer-Tropsch off gas drops to below a certain level, preferably in case the carbon monoxide level in the off gas is less than 20 vol %.

In fermentation processes the gaseous feedstock is important for providing appropriate conditions for the microbes to grow, to be maintained and to produce alcohol. The inventors have found that off gas obtained from a Fischer-Tropsch reaction is of sufficient quality to be used as a gaseous feedstock for fermentation. Preferably, the hydrogen:carbon monoxide ratio is at least 0.01. In a further aspect of the invention the hydrogen carbon monoxide ratio is at least 0.2 and in yet another aspect the ratio is at least 0.4. The off gas obtained from a Fischer-Tropsch reaction performed at industrial scale contains methane, carbon dioxide, carbon monoxide and hydrogen and optionally nitrogen, argon and traces of olefins and oxygenates. The levels of the different ingredients may depend on the hydrocarbonaceous feedstock used.

Hence, the method of the invention allows for the conversion of CO in off-gas into useful fermentation products such as ethanol. This improves the efficiency of the conversion of carbonaceous feedstock into useful products. Further by converting CO into useful products carbon dioxide emission is reduced.

In an aspect the off gas is obtained from a second or further Fischer-Tropsch reaction. This may be achieved by connecting two or more Fischer-Tropsch reactors in series.

In an aspect of the invention, the off gas of the fermentation process may be subjected to treatment step (a) of the process of the invention. This allows for an improvement in the efficiency of the process.

Depending on the stage of the process, i.e. growing microbes or producing alcohol, the content of the gaseous feedstock may be adapted. For example, prior to the start of the fermentation, microbes need to be multiplied/grown. At such a stage it may be beneficial to provide a gaseous feedstock having a high CO content. This may be achieved by providing off gas obtained from a first Fischer-Tropsch reaction, from syngas obtained from partial oxidation or a mixture thereof. It is to be noted that a too high a concentration of CO may lead to inhibition of fermentation or growth of the microbes.

In an aspect of the invention step (c) is performed in at least two bioreactors. In case of two or more reactors these reactors are preferably provided with off gas in parallel. This has as an advantage that off gas may still be processed by one reactor while the other reactor is taken offline. This may happen in case one bioreactor has to be provided with a new culture of microorganisms.

In an aspect of the invention the fermentation is affected by at least one member of the Carboxydotrophic bacteria such as Clostridium autoethanogenum.

In an aspect hydrogen is removed from the off gas prior to fermentation by means of pressure swing adsorption or after fermentation from a second off gas obtained in step (c). It is preferred that hydrogen is removed after fermentation. Hydrogen may be removed from the gas obtained from the fermentation process by use of pressure swing adsorption.

In an aspect of the invention a part of the gas mixture obtained in step (a) is subjected to anaerobic fermentation to obtain a fermentation product comprising alcohol. This may be useful in case the off gas generated in step (b) cannot sustain the microorganisms. This may be the case when the CO concentration and/or hydrogen concertation is too low. This may be the result of altered reaction circumstances for the Fischer-Tropsch reaction. This may occur during start-up or shut-down of a Fischer-Tropsch reactor.

In an aspect the fermentation product is subjected to distillation to separate at least the alcohol from the fermentation product. The raw liquid product exiting the reactor in which the fermentation is conducted is often referred to as broth. After the broth is obtained from the bioreactor part of the biomass is separated from the broth to obtain an alcohol-rich stream. The alcohol-rich stream is fed to a conventional alcohol separation setup containing two distillation columns and a molecular sieve unit (MSU) to produce fuel-grade alcohol. Advantageously, the energy generated by the Fischer-Tropsch reaction may be used in the distillation process.

The broth (a dilute acetic acid/nutrient solution) is then cooled to 37° C. before being returned to the reactor. Optionally nutrients and water are added to the broth before it is provided to the reactor. The water added to the broth may be demi water but is preferably process water obtained from other processes like the water generated by the Fischer-Tropsch reaction.

### DESCRIPTION OF THE DRAWINGS

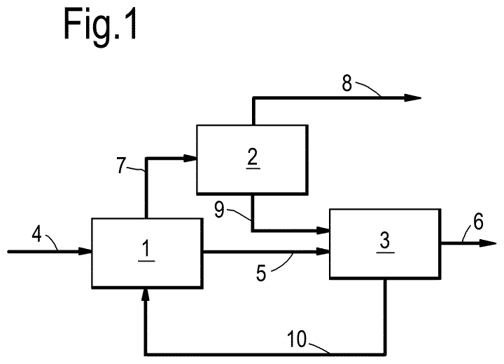

FIG. 1 shows a schematic representation of the present invention. The system depicted in FIG. 1 comprises a bioreactor 1, a scrubber 2 and an alcohol separation unit 3. Stream 4 represents a carbon monoxide and hydrogen containing gas mixture. Stream 4 is provided to the bioreactor 1 in which the carbon monoxide is converted into alcohol. Stream 4 has a pressure of 10 bar or less and preferably has a temperature of 40° C. or less and is preferably maintained between 35 and 40° C.

The bioreactor 1 contains microorganisms which facilitate the conversion process of carbon monoxide to alcohol. Alcohol is present in two streams leaving bioreactor 1. Namely, gaseous stream 7 (off gas) and a liquid stream 5 which is the fermentation broth. The broth exiting the reactor is filtered to remove microbes (not depicted). The alcohol-rich stream is fed to a conventional alcohol separation unit 3 containing two distillation columns and a molecular sieve unit (MSU) to produce fuel-grade alcohol 6.

The gaseous stream 7 exiting bioreactor 1 is fed to scrubber 2. Gaseous stream 7 may comprise alcohols, water and hydrogen and may be scrubbed to remove water and alcohol. The water and alcohol comprising stream leaving scrubber 2 may be fed together with or separately of the broth 5 to alcohol separation unit 3. Gaseous stream 8 leaving scrubber 2 may still contain hydrogen. Hence stream 8 may be provided to a pressure swing adsorption unit (not depicted) in order to obtain a hydrogen enriched gas stream.

The broth obtained after treatment in alcohol separation unit 3 may be provided to the bioreactor 1. Optionally, nutrients are provided to broth 10 prior to introduction of the broth to bioreactor 1.

While the invention has been described in terms of what are presently considered to be the most practical and preferred embodiments, it is to be understood that the disclosure need not be limited to the disclosed embodiments. It is intended to cover various modifications, combinations and similar arrangements included within the spirit and scope of the claims, the scope of which should be accorded the broadest interpretation so as to encompass all such modifications and similar structures. The present disclosure includes any and all embodiments of the following claims.

It should also be understood that a variety of changes may be made without departing from the essence of the invention. Such changes are also implicitly included in the description. They still fall within the scope of this invention.

Any patents, publications, or other references mentioned in this application for patent are hereby incorporated by reference. In addition, as to each term used, it should be understood that unless its utilization in this application is inconsistent with such interpretation, common dictionary definitions should be understood as incorporated for each term and all definitions, alternative terms, and synonyms such as contained in at least one of a standard technical dictionary recognized by artisans. One or more of the aspects of the invention may be combined and the appended claims form an integral part of this description by way of this reference.

1. A process for preparing a paraffin product from a carbonaceous feedstock comprising the following steps:
(a) treating the carbonaceous feedstock to obtain a gas mixture comprising hydrogen and carbon monoxide;
(b) performing at least once, a Fischer-Tropsch reaction using the gas mixture as obtained in step (a) and recovering a paraffin product and an off-gas comprising hydrocarbons, carbon dioxide, carbon monoxide, water and hydrogen from the Fischer-Tropsch reaction; and
(c) subjecting at least a part of the off-gas from the Fischer-Tropsch reaction to anaerobic fermentation to obtain a fermentation product comprising alcohol.

2. A process according to claim 1, wherein the Fischer-Tropsch reaction is conducted at elevated pressure and the process comprises a further step prior to step (c) wherein the off gas pressure is lowered to a pressure of less than 10 bar, prior to subjecting said off gas to fermentation.

3. A process according to claim 1, wherein a part of the gas mixture obtained in step (a) is subjected to anaerobic fermentation to obtain a fermentation product comprising alcohol.

4. A process according to claim 1, wherein the fermentation is affected by at least one member of the carboxydotrophic bacteria such as Clostridium autoethanogenum.

5. A process according to claim 1, wherein hydrogen is removed from the off gas prior to fermentation by means of pressure swing adsorption or after fermentation from a second off gas obtained in step (c).

6. A process according to claim 1, wherein the fermentation product is subjected to distillation to separate at least the alcohol from the fermentation product.

7. A process according to claim 1, wherein step (c) is performed in two or more bioreactors.

8. A process according to claim 1, wherein in step (a) the gas mixture is obtained by steam reforming and/or from the partial oxidation of natural gas.

9. A process according to claim 1, wherein in step (a) the gas mixture is obtained by partial combustion of coal.

10. A process according to claim 1, wherein the off-gas further comprises nitrogen, argon and traces of olefins and oxygenates.

11. A process according to claim 1, wherein subjecting at least a part of the off-gas from the Fischer-Tropsch reaction to anaerobic fermentation is conducted after removing hydrocarbons.
